(19) United States
(12) Patent Application Publication
(10) Pub. No.: US 2022/0168013 A1
(43) Pub. Date: Jun. 2, 2022

(54) STERILIZING SURGICAL ACCESS DEVICES

(71) Applicant: Covidien LP, Mansfield, MA (US)

(72) Inventors: Jacob C. Baril, Norwalk, CT (US); Garrett P. Ebersole, Hamden, CT (US); Kevin M. Desjardin, Prospect, CT (US); Douglas M. Pattison, East Hartford, CT (US); Christopher A. Tokarz, Wallingford, CT (US); Astley C. Lobo, West Haven, CT (US); Jason Mickus, Avon, CT (US)

(21) Appl. No.: 17/109,235

(22) Filed: Dec. 2, 2020

(51) Int. Cl. A61B 17/34; A61B 90/30

(52) U.S. Cl. CPC A61B 17/3423; A61B 2017/00902; A61B 90/30; A61B 17/3498

(57) ABSTRACT

A surgical access device sterilizes tissue at a surgical site and/or the air at the surgical site. The surgical access device may include a cannula including an elongated shaft having a fluid delivery channel defined therein, a fluid delivery port coupled to the cannula and in fluid communication with the fluid delivery channel, and a porous sleeve disposed around the elongated shaft and in fluid communication with the fluid delivery channel. The fluid delivery channel provides a pathway for fluid flow (e.g., an anti-infective agent) from the fluid delivery port into the porous sleeve. The surgical access device may include a cannula including an elongated shaft having an anti-infective agent disposed therein. The surgical access device may include an instrument housing secured to a cannula, and a light source (e.g., an air sanitizing light source) disposed within a cavity of the instrument housing.

### FIELD

This disclosure relates generally to surgical devices. In particular, the disclosure relates to surgical access devices positionable within tissue for sterilizing the tissue and/or the gases within a surgical site during a surgical procedure.

### BACKGROUND

In minimally invasive surgical procedures, including endoscopic and laparoscopic surgeries, a surgical access device permits the introduction of a variety of surgical instruments into a body cavity or opening. A surgical access device (e.g., a cannula or an access port) is introduced through an opening in tissue (e.g., a naturally occurring orifice or an incision) to provide access to an underlying surgical site in the body. The opening is typically made using an obturator having a blunt or sharp tip that may be inserted through a passageway of the surgical access device. For example, a cannula has a tube of rigid material with a thin wall construction, through which an obturator may be passed. The obturator is utilized to penetrate a body wall, such as an abdominal wall, or to introduce the surgical access device through the body wall, and is then removed to permit introduction of surgical instruments through the surgical access device to perform the surgical procedure.

Minimally invasive surgical procedures, including both endoscopic and laparoscopic procedures, permit surgery to be performed on organs, tissues, and vessels far removed from an opening within the tissue. In laparoscopic procedures, the abdominal cavity is insufflated with an insufflation gas, e.g., CO₂, to create a pneumoperitoneum thereby providing access to the underlying organs. A laparoscopic instrument is introduced through a cannula into the abdominal cavity to perform one or more surgical tasks. The cannula may incorporate a seal to establish a substantially fluid tight seal about the laparoscopic instrument to preserve the integrity of the pneumoperitoneum. The cannula, which is subjected to the pressurized environment, e.g., the pneumoperitoneum, may include an anchor mechanism to prevent the cannula from backing out of the opening in the abdominal wall, for example, during manipulation of the laparoscopic instrument within the cannula or withdrawal of the laparoscopic instrument therefrom. The cannula may also include a retention mechanism to prevent the cannula for being over-inserted into the abdominal wall, for example, during insertion of the laparoscopic instrument into the cannula. The retention mechanism generally utilizes mechanical interference with the cannula to create fixation. The holding force of the retention mechanism may be impacted during a surgical procedure by bodily fluids and/or surgical lubricants at the surgical site, manipulation of the cannula within the tissue during the surgical procedure, and/or multiple instrument insertions and withdrawals through the cannula.

During a surgical procedure, surgical personnel take precautions to prevent or reduce the risk of infection. Infection may be caused, for example, by the introduction of pathogens from the air, the surgical instruments used, and/or the patient's own tissue (e.g., tissue removed from the surgical site). This may lead to port site infections which can be painful and/or harmful to a patient's health.

Generally, sterilization techniques (e.g., sanitizing and/or disinfecting methods) are employed to limit contamination and minimize the risk of infection. Some techniques include spreading an antiseptic over the surgical site prior to a surgical procedure, using antibiotics or other bacteria growth inhibitors prior to or during the surgical procedures, and/or cleaning the port site after the surgical procedure is complete.

### SUMMARY

This disclosure generally relates to surgical access devices designed to sterilize (e.g., disinfect, decontaminate, sanitize, clean, and/or purify) tissue at a surgical site and/or the gases within the surgical site during a surgical procedure. The surgical access devices of the disclosure include cannulas that are positioned within tissue and treat the tissue (e.g., the tissue wall at the port site and/or the tissue within the body cavity) and/or the gases disposed within the body cavity. The surgical access devices minimize infections (e.g., port site infections and/or transmittance of airborne pathogens) and increase the effectiveness of combating and/or preventing infection during a surgical procedure.

In one aspect, this disclosure provides a surgical access device including a cannula including an elongated shaft having a fluid delivery channel defined therein, a fluid delivery port coupled to the cannula and in fluid communication with the fluid delivery channel, and a porous sleeve disposed around the elongated shaft and in fluid communication with the fluid delivery channel. The fluid delivery channel provides a pathway for fluid flow from the fluid delivery port into the porous sleeve.

The surgical access device may further include a fluid source coupled to the fluid delivery port. The fluid source may include an anti-infective agent. The anti-infective agent may be an antibiotic.

The fluid delivery port may extend longitudinally along an outer surface of the elongated shaft. A proximal end of the fluid delivery channel may be disposed within the fluid delivery port. A proximal portion of the porous sleeve may be positioned distal to the proximal end of the fluid delivery channel and a distal portion of the porous sleeve may be positioned distal to a distal end of the fluid delivery channel.

The porous sleeve may be a foam.

In another aspect, this disclosure provides a surgical access device including a cannula including an elongated shaft having an anti-infective agent disposed therein and an instrument housing secured to the cannula.

The anti-infective agent may be silver. The anti-infective agent may be impregnated into the elongated shaft of the cannula.

The surgical access device may further include a retention collar supported on the cannula. The retention collar may include a tissue facing surface including an adhesive and/or an anti-infective agent disposed thereon.

In yet another aspect, this disclosure provides a surgical access device including a cannula having an elongated shaft and an instrument housing secured to the cannula. The instrument housing defines a cavity therein and includes a light source disposed within the cavity.

The elongated shaft may include an inner tube and an outer tube coaxially mounted over the inner tube. The inner tube may be formed from a transparent material and the outer tube may be formed from an opaque material.

The light source may emit ultraviolet light. The light source may include a plurality of light emitting elements equidistantly spaced from each other circumferentially about the cavity.

The instrument housing may include a valve assembly disposed within the cavity and the light source may be disposed distal to the valve assembly.

The instrument housing may include a power pack coupled thereto. The power pack may include a power source and a circuit board. The power pack may be electrically coupled to the light source to power the light source.

The details of one or more aspects of the disclosure are set forth in the accompanying drawings and the description below. Other features, objects, and advantages of the aspects described in this disclosure will be apparent from the description and drawings, and from the claims.

### BRIEF DESCRIPTION OF THE DRAWINGS

FIG. 1 is a perspective view of a surgical access assembly including a surgical access device in accordance with an aspect of the disclosure;

FIG. 2 is a perspective view of the surgical access assembly of FIG. 1, shown with a porous sleeve separated from a cannula of the surgical access device;

FIG. 3 is cross-sectional view of the surgical access assembly of FIG. 1, taken along section line 3-3 of FIG. 1;

FIG. 4 is a cross-sectional view of the surgical access assembly of FIG. 1, taken along section line 4-4 of FIG. 1, shown secured to tissue;

FIG. 5 is a side view of a surgical access assembly in accordance with another aspect of the disclosure, shown secured to tissue;

FIG. 6 is a perspective view of a surgical access device in accordance with an aspect of the disclosure;

FIG. 7 is a cross-sectional view of the surgical access device of FIG. 6, taken along section line 7-7 of FIG. 6;

FIG. 8 is a perspective view of a surgical access assembly in accordance with another aspect of the disclosure; and

FIG. 9 is a side view of the surgical access assembly of FIG. 8, shown secured to tissue.

### DETAILED DESCRIPTION

Aspects of this disclosure are described hereinbelow with reference to the accompanying drawings; however, it is to be understood that the disclosed aspects are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure.

The surgical access assemblies of this disclosure will be described to the extent necessary to disclose aspects of the disclosure. For a detailed description of the structure and function of components of exemplary surgical access assemblies, reference may be made to U.S. Pat. Nos. 7,300,448; 7,691,089; and 8,926,508, the entire content of each of which is hereby incorporated by reference herein.

Surgical access assemblies with obturators, known as trocar assemblies, are employed during minimally invasive surgery, e.g., laparoscopic surgery, and provide for the sealed access of surgical instruments into an insufflated body cavity, such as the abdominal cavity. The surgical access assemblies of the disclosure include a surgical access device having an instrument housing mounted on a cannula. An obturator (not shown) is insertable through the instrument housing and the cannula. The handle of the obturator can engage or selectively lock into the instrument housing of the surgical access device. The obturator can have a blunt distal end, or a bladed or non-bladed penetrating distal end, and can be used to incise and/or separate tissue of the abdominal wall so that the surgical access assembly can be introduced into the abdomen. The obturator can be optical to provide contemporaneous visualization of body tissue fibers as they are being separated.

Trocar obturators suitable for use with the surgical access devices of the disclosure are known and include, for example, bladed, bladeless, blunt, optical, and non-optical. For a detailed description of the structure and function of exemplary trocar assemblies, including exemplar trocar obturators, reference may be made to PCT Publication No. WO 2016/186905, the entire content of which is hereby incorporated by reference herein.

Like reference numerals refer to similar or identical elements throughout the description of the figures. Throughout this description, the term “proximal” refers to a portion of a structure, or component thereof, that is closer to a user, and the term “distal” refers to a portion of the structure, or component thereof, that is farther from the user.

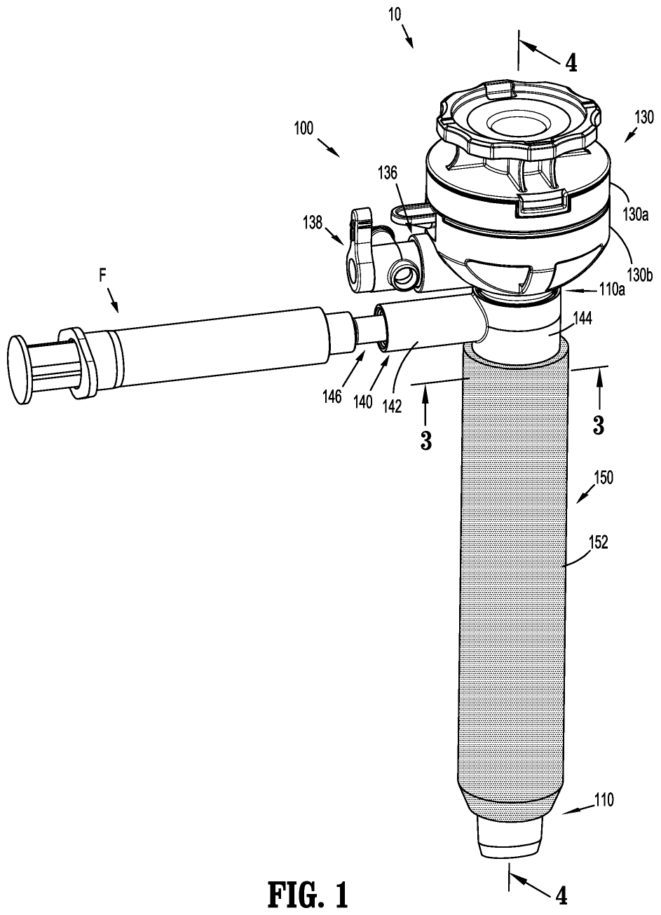

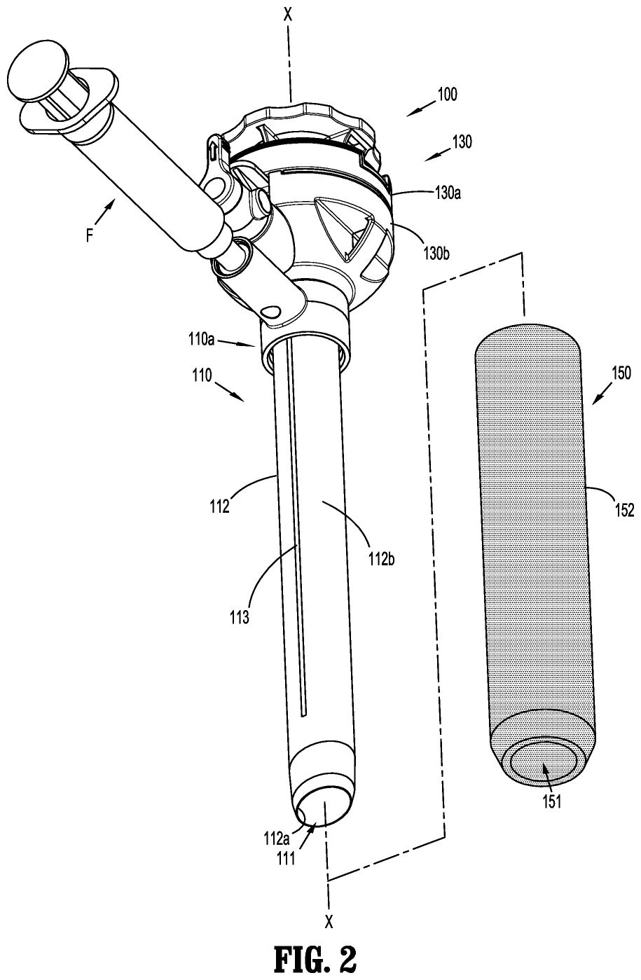

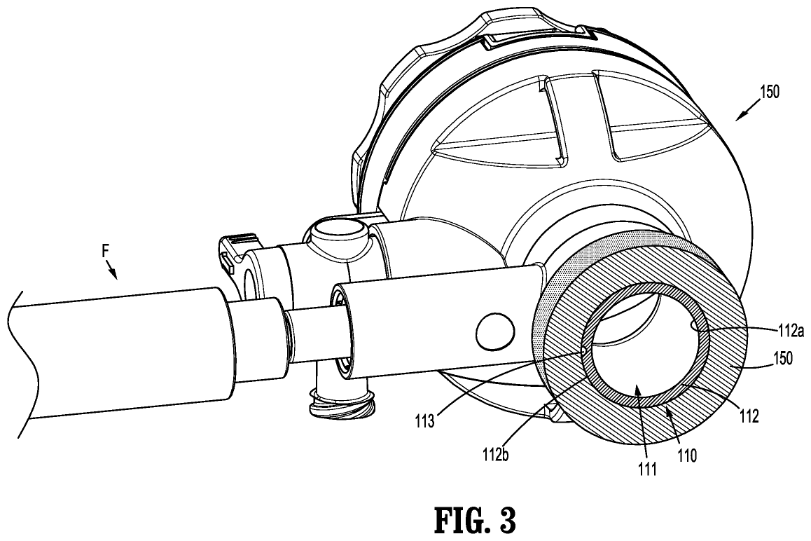

FIGS. 1-4 illustrate a surgical access assembly 10 in accordance with an aspect of the disclosure. The surgical access assembly 10 includes a surgical access device 100 and a fluid source “F” couplable to the surgical access device 100. The surgical access device 100 includes a cannula 110, an instrument housing 130 secured to the cannula 110, and a porous sleeve 150 disposed on the cannula 110. The cannula 110 includes an elongated shaft 112 extending along a longitudinal axis “X” (FIG. 2). As seen in FIGS. 2 and 3, the elongated shaft 112 includes an inner surface 112a defining an access lumen 111 for reception and passage of a surgical instrument (not shown) therethrough, and an outer surface 112b defining a fluid delivery channel 113 therein that extends longitudinally along a length (e.g., a majority of the length) of the elongated shaft 112.

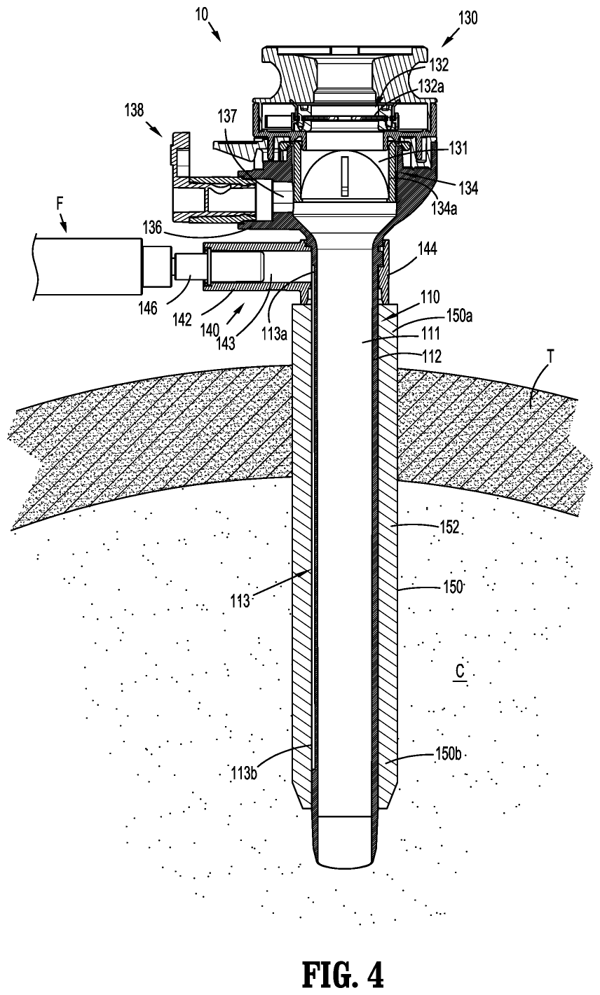

With continued reference to FIGS. 1-4, a proximal end portion 110a of the cannula 110 supports the instrument housing 130 thereon. The instrument housing 130 includes an upper housing section 130a and a lower housing section 130b, and defines a cavity 131 (FIG. 4) therein that communicates with the access lumen 111 of the elongated shaft 112 of the cannula 110. The upper housing section 130a may be selectively attachable to, and detachable from, the lower housing section 130b, and the lower housing section 130b may be releasably or permanently attached to the elongated shaft 112 of the cannula 110. In aspects, either or both of the upper and lower housing sections 130a, 130b of the instrument housing 130 may include knurls, indentations, tabs, or be otherwise configured to facilitate engagement by a user.

As seen in FIG. 4, the instrument housing 130 supports a seal assembly 132 and a valve assembly 134 therein. The seal assembly 132 is disposed proximally of the valve assembly 134. The seal assembly 132 generally includes an instrument seal 132a for sealing around surgical instruments (not shown) inserted into the cannula 110, and the valve assembly 134 generally includes a zero-closure seal 134a for sealing the access lumen 111 of the cannula 110 in the absence of a surgical instrument inserted through the cannula 110. The seal assembly 132 and the valve assembly 134 prevent the escape of the insufflation fluid therefrom, while allowing surgical instruments to be inserted therethrough and into the body cavity. The instrument seal 132a may include any known instrument seal used in cannulas and/or trocars, such as a septum seal. The zero-closure seal 134a may be any known zero-closure seal for closing off the passageway into the access lumen 111, such as a duckbill seal or a flapper valve.

The instrument housing 130 includes an insufflation port 136 coupled to the lower housing section 130b. The insufflation port 136 defines an opening 137 therethrough that is in fluid communication with the cavity 131 of the instrument housing 130 which, in turn, is in fluid communication with the access lumen 111 of the cannula 110 to insufflate a body cavity, such as abdominal cavity (e.g., create a pneumoperitoneum). The opening 137 of the insufflation port 136 is disposed distally of the valve assembly 134 to maintain insufflation pressure within the body cavity. The insufflation port 136 is connectable to a source of insufflation fluid (not shown) for delivery of the insufflation fluid (e.g., gases) into the body cavity. The insufflation port 136 is configured and dimensioned to receive a valve 138 in a substantially fluid-tight manner. In aspects, and as shown, the valve 138 is a stopcock valve for controlling the flow of the insufflation fluid. The valve 138, however, may be any known valve for directing fluid flow and, in some aspects, regulating fluid flow.

The surgical access device 100 includes a fluid delivery port 140 coupled to the elongated shaft 112 of the cannula 110. The fluid delivery port 140 includes a housing 142 and a collar 144 extending from the housing 142. The collar 144 secures the housing 142 to the elongated shaft 112 of the cannula 110. The collar 144 extends around the elongated shaft 112 and is engaged with the elongated shaft 112 in a manner that fixes (e.g., longitudinally and rotationally) the fluid delivery port 140 relative to the cannula 110. More particularly, the collar 144 may be engaged with the elongated shaft 112 by snap fit connection, ultrasonic welding, or in a friction fit manner. It should be understood that other mating structures and relationships may be utilized to secure the fluid delivery port 140 to the elongated shaft 112.

The fluid delivery port 140 is in fluid communication with the fluid delivery channel 113 defined in the elongated shaft 112. The fluid delivery port 140 is connectable to the fluid source “F” and defines an opening 143 therein for delivery of a fluid (e.g., liquid) into the fluid delivery channel 113 through the housing 142. A proximal end 113a of the fluid delivery channel 113 is disposed within the opening 143 of the housing 142 and in fluid communication therewith to provide an inlet into the fluid delivery channel 113. The fluid delivery port 140 includes a valve 146 operably coupled to the housing 142 in a substantially fluid-tight manner for controlling fluid flow therethrough. In aspects, the fluid source “F” is a syringe coupled to the valve 146 (e.g., a tip of the syringe is positioned within the valve) to deliver fluid into the fluid delivery channel 113.

The fluid source “F” contains a fluid having one or more anti-infective agents. The anti-infective agents may be antimicrobials, antibacterials, antibiotics, antivirals, antiseptics, antifungals, among other agents for treating or inhibiting infection. Additionally or alternatively, bioactive agents (for example, substances or mixtures of substances having clinical use, such as those providing a therapeutic or prophylactic effect or play a role in one or more biological processes) may be introduced through the fluid source “F”.

The porous sleeve 150 has an elongated body 152 defining a lumen 151 (FIG. 2) therethrough. The lumen 151 is sized and shaped to receive the elongated shaft 112 of the cannula 110 therein such that the porous sleeve 150 surrounds the elongated shaft 112. The porous sleeve 150 is releasably engaged with the elongated shaft 112 (e.g., slidable onto and off of the elongated shaft 112). The porous sleeve 150 may be shaped to mimic the shape of the outer surface 112b of the elongated shaft 112. In aspects, the porous sleeve 150 is substantially tubular, however, it should be understood that the shape of the porous sleeve 150 may vary. As seen in FIG. 4, a proximal portion 150a of the porous sleeve 150 is disposed distal to the proximal end 113a of the fluid delivery channel 113 and a distal portion 150b of the porous sleeve 150 covers and extends distal to a distal end 113b of the fluid delivery channel 113.

The porous sleeve 150 may be compressible and capable of undergoing a change in shape during positioning in tissue. The porous sleeve 150 may compress during insertion into tissue and expand once placed in the tissue (e.g., from heat or fluid contact) to seal the port site and/or limit movement of the cannula 110 relative to the tissue. The porous sleeve 150 may be formed from a porous material having pores or perforations designed to soak up and release fluid. In aspects, the porous sleeve 150 is formed from a foam. The foam may have an open cell structure where pores are connected to each other, forming an interconnected network. Conversely, the foam may be a closed cell foam where the pores are not interconnected. Closed cell foams are generally denser and have a higher compressive strength. Characteristics of the porous sleeve 150 (e.g., material, durometer, porosity, thickness, etc.) may be selected to control the elution rate of a fluid therefrom. In some aspects, the porous sleeve 150 is configured to achieve a sustained, controlled release of fluid therefrom. Accordingly, the porous sleeve 150 provides fixation within tissue as well as acts as a vehicle for drug delivery into the tissue and thus, provides a safe, easy, and effective means for drug delivery to a surgical site without sacrificing device fixation.

FIG. 4 illustrates the surgical access assembly 10 disposed within tissue “T,” e.g., an abdominal wall. In a method of use, the elongated shaft 112 of the cannula 110, having the porous sleeve 150 positioned thereon, is received through the tissue “T” (e.g., by utilizing an obturator (not shown) to facilitate entry of the cannula 110 through the tissue “T”) such that the porous sleeve 150 is positioned adjacent to the tissue “T” (e.g., the tissue at the incision or port site). As described above, the porous sleeve 150 seals the tissue “T” and aids in retaining the elongated shaft 112 within the tissue “T” to minimize longitudinal movement of the cannula 110 relative to the tissue “T,” for example, during withdrawal and/or manipulation of a surgical instrument (not shown) through the cannula 110.

The fluid source “F” is coupled to the fluid delivery port 140 and the fluid containing the anti-infective agent disposed within the fluid source “F” is delivered into the porous sleeve 150 and distributed evenly throughout the porous sleeve 150. Specifically, as described above, the fluid delivery channel 113 provides a pathway for the fluid from the fluid source “F” into the porous sleeve 150. The fluid is distributed by passing the fluid from the fluid source “F” into the fluid delivery port 140, through the fluid delivery channel 113, and into the porous sleeve 150. The fluid and thus, the anti-infective agent, is evenly dispersed into the tissue “T” and the body cavity “C” as it is released from the porous sleeve 150. In some aspects, the porous sleeve 150 allows the continuous elution of the fluid into the tissue “T” throughout the surgical procedure. Accordingly, surgical personnel can control the release of the anti-infective agent into and throughout the tissue “T” at the surgical site thereby minimizing or preventing port site infections while maintaining device fixation.

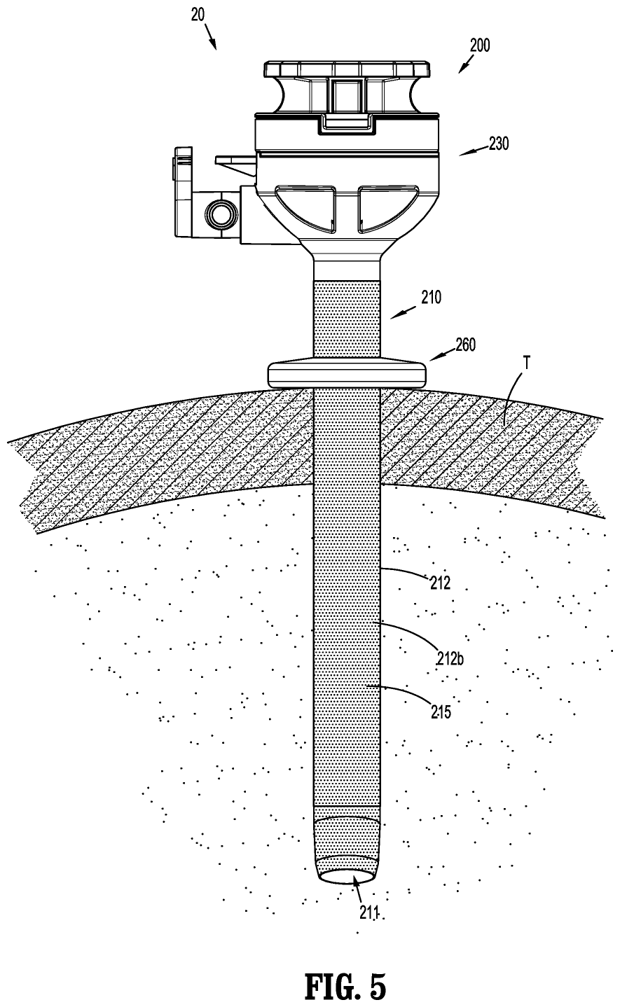

Turning now to FIG. 5, a surgical access assembly 20 in accordance with another aspect of the disclosure is shown. The surgical access assembly 20 includes a surgical access device 200 and a retention collar 260. The surgical access device 200 includes a cannula 210 and an instrument housing 230 secured to the cannula 210. The retention collar 260 is disposed on the cannula 210. The instrument housing 230 is substantially the same as the instrument housing 130 (e.g., the instrument housing 230 includes upper and lower housing sections, a seal assembly, a valve assembly, and an insufflation port).

The cannula 210 generally includes an elongated shaft 212 defining an access lumen 211 for reception and passage of a surgical instrument (not shown) therethrough. The elongated shaft 212 includes one or more anti-infective agents 215 disposed in at least an outer surface 212b of the elongated shaft 212. The anti-infective agents 215 may be antimicrobials, antibacterials, antibiotics, antivirals, antiseptics, antifungals, among other agents for treating or inhibiting infection. Suitable antimicrobial agents include, but are not limited to, ionic metals, silver and silver compounds, zinc, copper, and combinations thereof. In some aspects, the anti-infective agent 215 uses water (e.g., bodily fluid) to transport media, such as silver. The elongated shaft 212 may also include additional bioactive agents.

The anti-infective agent 215 may be combined directly (e.g., impregnated or embedded) with the material forming the elongated shaft 212. In aspects, the anti-infective agent is an antimicrobial, such as silver, that is embedded into a molded cannula 210 and eludes into tissue “T” during a surgical procedure to treat (e.g., prevent) port site infection. In some aspects, the cannula 210 may also have added strength due to the inclusion of the anti-infective agent 215, such as silver, in the elongated shaft 212. The anti-infective agent 215 may be uniformly and homogenously combined with the material forming the elongated shaft 212, or may be provided in a desired concentration or concentration gradient to portion(s) or the entirety of the elongated shaft 212. Alternatively, the anti-infective agent 215 may be applied to the elongated shaft 212, for example, in a single layer or multiple layers, or as a composition disposed thereon. The anti-infective agent 215 may be contained and subsequently released by a delivery agent (e.g., a hydrogel coating, a dissolvable film, etc.).

The retention collar 260 is supported on the elongated shaft 212 of the cannula 210. The retention collar 260 is releasably engageable with the elongated shaft 212, and slidable therealong to adjust the longitudinal position of the retention collar 260 on the elongated shaft 212. The retention collar 260 is configured to mechanically (e.g., frictionally) engage the elongated shaft 212 to limit movement of the retention collar 260 relative to the cannula 210 and to secure the cannula 210 against tissue “T” (e.g., an outer surface of a body wall). The retention collar 260 may be formed from a compressible material (e.g., foam, cotton, or other suitable textile) to aid in sealing the opening into the tissue “T” of the body wall (e.g., the port site). The retention collar 260 may include any known retention mechanism used on cannulas and/or trocars, such as a rubber donut or a foam collar.

As seen in FIG. 5, the surgical access assembly 20 disposed within tissue “T,” e.g., an abdominal wall. In a method of use, the shaft assembly 212 of the cannula 210 is received through the tissue “T” (e.g., by utilizing an obturator (not shown) to facilitate entry of the cannula 210 through the tissue “T”) such that the anti-infective agent 215 contacts the tissue “T” and eludes into the tissue “T” throughout the surgical procedure to help reduce the occurrence of port site infection. The retention collar 260 is slid distally along the elongated shaft 212 of the cannula 210 until the retention collar 260 abuts or presses on the tissue “T” to aid in retaining the cannula 210 in position within the tissue “T”.

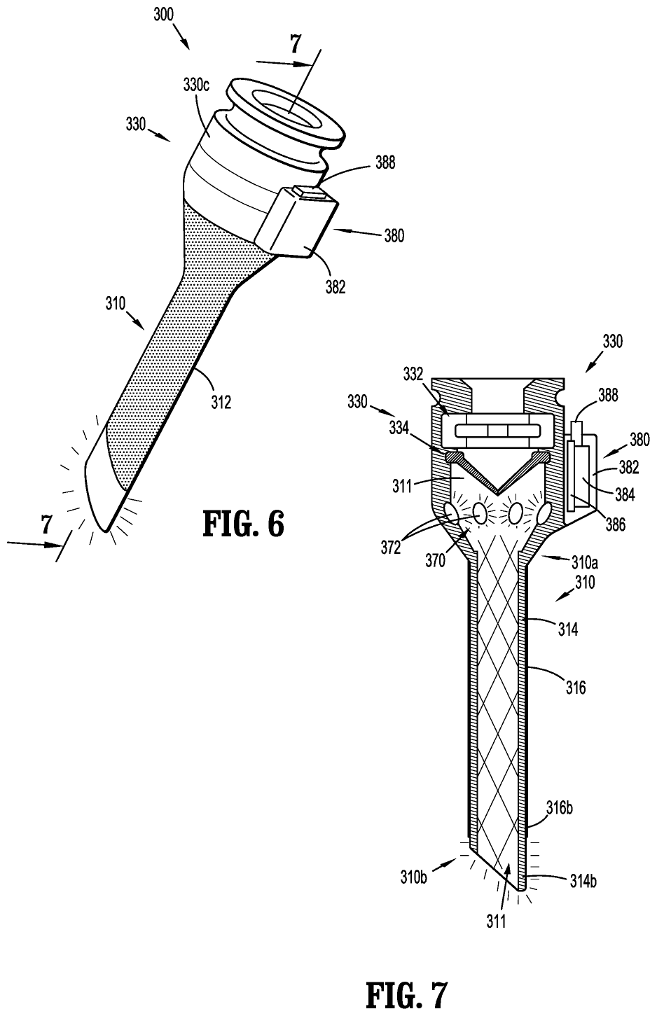

Referring now to FIGS. 6 and 7, a surgical access device 300 in accordance with another aspect of the disclosure is shown. The surgical access device 300 includes a cannula 310 and an instrument housing 330 secured to the cannula 310. The surgical access device 300 includes a light source 370 (e.g., a UV light source) in the instrument housing 330 that sterilizes the gas (e.g., air, etc.) traveling the length of the cannula 310 (e.g., gas going into and out of a body cavity).

The cannula 310 generally includes an elongated shaft 312 defining an access lumen 311 for reception and passage of a surgical instrument (not shown) therethrough. The elongated shaft 312 includes an inner tube 314 and an outer tube 316 coaxially mounted over the inner tube 314. The inner tube 314 is a light pipe for transmitting light along the length thereof. In aspects, the inner tube 314 is constructed of a transparent material (e.g., a polymeric material), however, other constructions (e.g., the incorporation of reflective materials or optical fibers) are envisioned. The outer tube 316 sheaths the inner tube 314 and masks the light emitted therethrough to prevent damage to the surrounding tissue at the port site (e.g., UV damage). In aspects, the outer tube 316 is constructed of an opaque material (e.g., a polymeric material).

The outer tube 316 covers substantially the entirety of the inner tube 314. A distal end portion 314b of the inner tube 314 may extend distally beyond a distal end portion 316b of the outer tube 316 such that the cannula 310 provides ambient light at the distal end portion 310b thereof. In some aspects, the ambient light supplements the light emitted from an endoscope positioned at the surgical site and/or reduces the required lighting intensity of the endoscope while limiting image washout. Alternatively, the outer tube 316 may cover the entirety of the inner tube 314 and, in some aspects, a light source (e.g., a natural light source) may be provided at the distal end portion 310b of the cannula 310 to illuminate the body cavity in which the cannula 310 is disposed.

A proximal end 310a of the cannula 310 supports the instrument housing 330 thereon. The instrument housing 330 is substantially similar to instrument housing 130, 230 and will be described with respect to the differences therebetween. The instrument housing 330 defines a cavity 331 therein that communicates with the access lumen 311 of the elongated shaft 312 of the cannula 310. The instrument housing 330 supports a seal assembly 332 and a valve assembly 334 therein, and may include an insufflation port (not shown) for insufflating a body cavity. The light source 370 is disposed within the cavity 331 of the instrument housing 330 distal to the valve assembly 334. The light source 370 emits ultraviolet light for disinfecting the gases and preventing transmission of a variety of airborne infections (e.g., gases entering and/or exiting the body cavity). In aspects, the light source 370 includes a plurality of light emitting elements 372 and, in some aspects, the light emitting elements 372 are light emitting diodes. The light emitting element 372 are equidistantly spaced from each other circumferentially about the cavity 331 to transmit light evenly into the cavity 331. Other configurations of the light source 370, however, are envisioned.

The instrument housing 330 includes a power pack 380 extending from an outer surface 330c thereof. The power pack 380 includes a casing 382 housing a power source 384 (e.g., a battery) and a circuit board 386 (e.g., a printed circuit board) therein, and having an actuator 388 (e.g., a button or a switch) thereon. The light source 370 is electrically coupled to the circuit board 386, which is electrically coupled to the power source 384 and the actuator 388, such that the light source 370 is powered by the power source 384 and activated (e.g., on and off) by the actuator 388. The power pack 380 may be releasably coupled to the instrument housing 330 for proper disposal or for recharging and re-use.

In use, upon actuation of the actuator 388 to power on the light source 370, the light source 370 illuminates the cavity 331 within the instrument housing 330 with ultraviolet light which also travels the length of the cannula 110 within the inner tube 314 of the elongated shaft 312 thereby sterilizing the gas as it passes through the instrument housing 330 and the cannula 110 (e.g., during instrument withdrawal and/or exchange) to reduce the potential release of airborne pathogens, such as COVID-19, into the atmosphere in the operating room (e.g., gas released during surgery that escapes through the cannula). The ultraviolet light is also slightly emitted through the distal end portion 310b of the cannula 310 to help sterilize the gas within the body cavity in which the cannula 310 is disposed. In certain aspects, the elongated shaft 312 may be formed of conventional materials such that the light source 370 mainly emits within the cavity 331 of the instrument housing 330 and sterilizes the air as it travels therethrough.

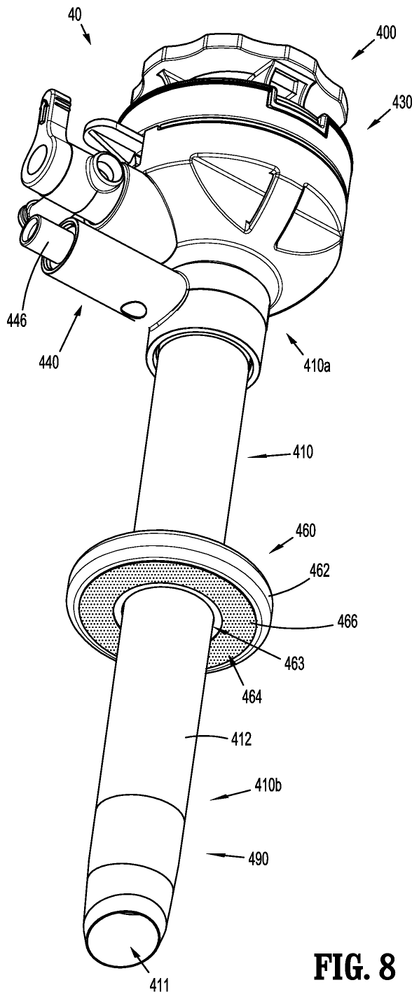

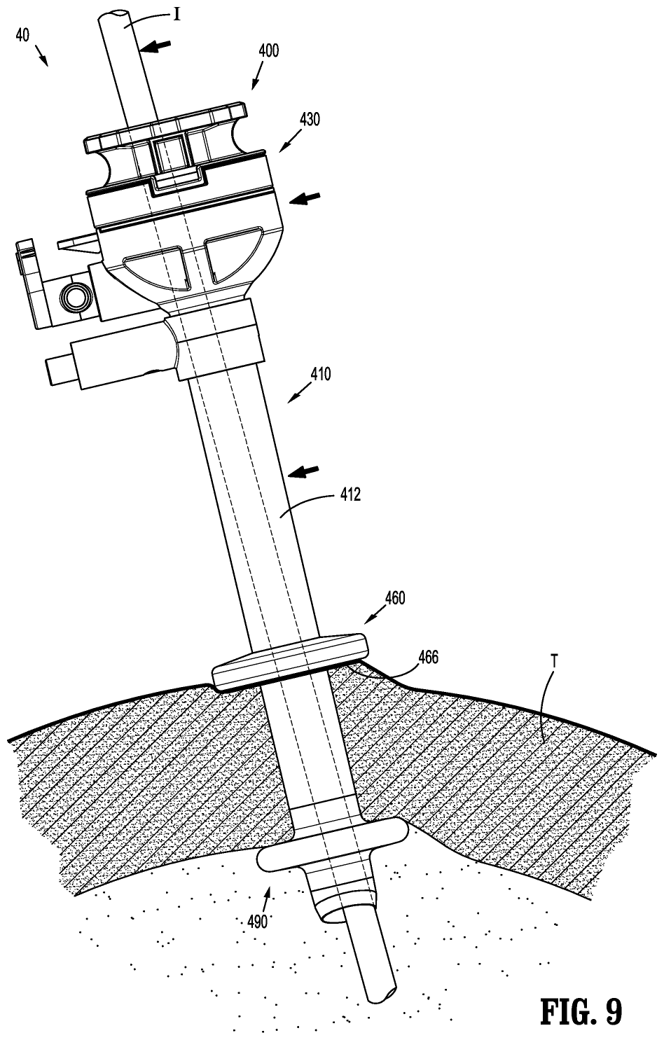

FIGS. 8 and 9 illustrate a surgical access assembly 40 including a cannula 410 having fixation devices 460, 490 for securing the cannula 410 within tissue “T.” The fixation devices 460, 490 provide fixation on both sides of a tissue wall (e.g., fixation in two directions during instrument removal and insertion).

The surgical access assembly 40 includes a surgical access device 400 including the cannula 410 and an instrument housing 430 secured to the cannula 410, and a retention collar 460 disposed around the cannula 410. The cannula 410 generally includes an elongated shaft 412 defining an access lumen 411 for reception and passage of a surgical instrument “I” therethrough. A proximal end portion 410a of the cannula 410 supports the instrument housing 430 thereon. The instrument housing 430 is substantially the same as the instrument housing 130, 230 (e.g., the instrument housing 430 includes upper and lower housing sections, a seal assembly, a valve assembly, and an insufflation port). A distal end portion 410b of the cannula 410 supports an expandable anchor 490. The expandable anchor 490 is secured (e.g., glued and/or welded) to the elongated shaft 412 to create hermetic contact therebetween, or may be formed (e.g., blow molded) as a single piece with the elongated shaft 412. The expandable anchor 490 secures the cannula 410 against tissue “T” (e.g., an inner surface of a body wall). While the expandable anchor 490 is shown as an inflatable anchor (e.g., a balloon), it should be understood that other fixation structures, such as a contractable anchor (e.g., a collapsible flange) may be utilized as a fixation device 490.

The cannula 410 includes a fluid delivery port 440 that is substantially the same as the fluid delivery port 140 but is in fluid communication with the expandable anchor 490 and includes a valve 446 for controlling the flow of fluid into and out of the expandable anchor 490. The cannula 410 includes an inflation lumen or passageway (not explicitly shown) defined therethrough that is in fluid communication with the fluid delivery port 440 and the expandable anchor 490. In some aspects, the inflation lumen is similar in construction to the fluid delivery channel 113 (FIG. 4) and covered by a fixed sleeve portion of the expandable anchor 380 or additional structure of the elongated shaft 412 to form a discrete fluid flow pathway from the fluid delivery port 440 to the expandable anchor 490.

To inflate the expandable anchor 490, a fluid source (not shown) is releasably attached to the fluid delivery port 440 and pressurized fluid is introduced into the fluid delivery port 440, through the inflation lumen, and into the expandable anchor 490 causing the expandable anchor 490 to expand (FIG. 9). To deflate the expandable anchor 490, the fluid is allowed to escape through the fluid delivery port 440 thereby causing the expandable anchor 490 to retract or collapse (FIG. 8).

The retention collar 460 is supported on the elongated shaft 412 of the cannula 410. The retention collar 460 includes an annular body 462 having an opening 463 defined therethrough that is sized and shaped to accommodate the elongated shaft 412 of the cannula 410 therein. The retention collar 460 is releasably engageable with elongated shaft 412, and slidable therealong to adjust the longitudinal position of the retention collar 460 on the elongated shaft 412. The retention collar 460 is configured to mechanically (e.g., frictionally) engage the elongated shaft 412 to limit movement of the retention collar 460 relative to the cannula 410 and to secure the cannula 410 against tissue “T” (e.g., an outer surface of a body wall).

The retention collar 460 includes a tissue contacting surface 464 that faces distally when the retention collar 460 is positioned on the cannula 410. The tissue contacting surface 464 includes an adhesive 466 disposed thereon that is configured to adhere and bond to tissue “T”. The adhesive 466 is biocompatible and may be in the form of a sticker secured to the tissue contacting surface 464 of the retention collar 460 or a composition applied to the tissue contacting surface 464. In aspects, the adhesive 466 is fluid activated (e.g., saline or bodily fluids) and/or may include a removable release liner disposed thereover until the time of application.

The retention collar 460 helps seal the opening in the tissue “T” of the body wall (e.g., the port site), and creates fixation through mechanical and chemical engagement of the retention collar 460 to the elongated shaft 412 and the tissue “T,” respectively. The adhesive 466 further helps prevent the loss of fixation by ensuring the retention collar 460 stays in contact with the tissue “T” and the retention collar 460 can withstand more force as compared to retention collars that only utilize mechanical engagement between the retention collar and the cannula.

In some aspects, the tissue contacting surface 464 of the retention collar 460 may additionally or alternatively include an anti-infective agent disposed thereon to aid in minimizing the occurrence of port site infection. The anti-infective agent may be antimicrobials, antibacterials, antibiotics, antivirals, antiseptics, antifungals, among other agents for treating (e.g., preventing) infection. The anti-infective agent may be imbedded or impregnated into the tissue contacting surface 464 or applied thereon (e.g., as a film or coating). In certain aspects, the anti-infective agent may be combined with the adhesive 466.

FIG. 9 illustrates the surgical access assembly 40 disposed within tissue “T,” e.g., an abdominal wall. The elongated shaft 412 of the cannula 410 is received through the tissue “T” (e.g., by utilizing an obturator (not shown) to facilitate entry of the cannula 410 through the tissue “T”), and the expandable anchor 490 is inflated within a body cavity “C” to prevent the cannula 410 from being withdrawn through the tissue “T.” The retention collar 460 is slid distally along the elongated shaft 412 of the cannula 410 until the retention collar 460 abuts or presses on the tissue “T” and the adhesive 466 bonds to the tissue “T.” The tissue “T” is thus sandwiched between the expandable anchor 490 and the retention collar 460, with the retention collar 460 bonded to the tissue “T,” to prevent the cannula 410 from being withdrawn from or over-inserted into the tissue “T.” The retention collar 460 maintains the position of the cannula 410 within the tissue “T” even if the cannula 410 is disposed at an angle with respect to the tissue “T,” or the cannula 410 or a surgical instrument “I” inserted therethrough is subjected to a lateral force. In this manner, the surgical access assembly 40 is secured to the tissue “T” and movement of the cannula 410 relative to the tissue “T” is prevented or minimized throughout insertion, withdrawal, and/or manipulation of a surgical instrument (not shown) through the cannula 110. Following the surgical procedure, the expandable anchor 490 is deflated to permit the withdrawal of the surgical access assembly 40 from the tissue “T.”

It should be understood that the surgical access devices 100, 200, 300 may include the fixation devices 460, 490. For example, the elongated shafts 112, 212, 312 may support a retention collar 460 thereon and/or the distal end portions of the cannulas 110, 210, 310 may support an expandable anchor 490 thereon.

While aspects of the disclosure have been described and shown in the drawings, it is not intended that the disclosure be limited thereto, as it is intended that the disclosure be as broad in scope as the art will allow and that the specification be read likewise. It is to be understood, therefore, that the disclosure is not limited to the precise aspects described, and that various other changes and modifications may be effected by one skilled in the art without departing from the scope or spirit of the disclosure. Additionally, the elements and features shown and described in connection with certain aspects of the disclosure may be combined with the elements and features of certain other aspects without departing from the scope of the disclosure, and that such modifications and variation are also included within the scope of the disclosure. Therefore, the above description should not be construed as limiting, but merely as exemplifications of aspects of the disclosure. Thus, the scope of the disclosure should be determined by the appended claims and their legal equivalents, rather than by the examples given.

What is claimed is:

1. A surgical access device comprising:
a cannula including an elongated shaft having a fluid delivery channel defined therein;
a fluid delivery port coupled to the cannula and in fluid communication with the fluid delivery channel; and
a porous sleeve disposed around the elongated shaft and in fluid communication with the fluid delivery channel,
the fluid delivery channel providing a pathway for fluid flow from the fluid delivery port into the porous sleeve.

2. The surgical access device according to claim 1, further including a fluid source coupled to the fluid delivery port, the fluid source including an anti-infective agent.

3. The surgical access device according to claim 2, wherein the anti-infective agent is an antibiotic.

4. The surgical access device according to claim 1, wherein the fluid delivery port extends longitudinally along an outer surface of the elongated shaft.

5. The surgical access device according to claim 4, wherein a proximal end of the fluid delivery channel is disposed within the fluid delivery port.

6. The surgical access device according to claim 5, wherein a proximal portion of the porous sleeve is distal to the proximal end of the fluid delivery channel and a distal portion of the porous sleeve is distal to a distal end of the fluid delivery channel.

7. The surgical access device according to claim 1, wherein the porous sleeve is a foam.

8. A surgical access device comprising:
a cannula including an elongated shaft having an anti-infective agent disposed therein; and
an instrument housing secured to the cannula.

9. The surgical access device according to claim 8, wherein the anti-infective agent is silver.

10. The surgical access device according to claim 8, wherein the anti-infective agent is impregnated into the elongated shaft of the cannula.

11. The surgical access device according to claim 8, further including a retention collar supported on the cannula.

12. The surgical access device according to claim 11, wherein the retention collar includes a tissue facing surface including an adhesive disposed thereon.

13. The surgical access device according to claim 11, wherein the retention collar includes a tissue facing surface including an anti-infective agent disposed thereon.

14. A surgical access device comprising:
a cannula including an elongated shaft; and
an instrument housing secured to the cannula, the instrument housing defining a cavity therein and including a light source disposed within the cavity.

15. The surgical access device according to claim 14, wherein the elongated shaft includes an inner tube and an outer tube coaxially mounted over the inner tube.

16. The surgical access device according to claim 15, wherein the inner tube is formed from a transparent material and the outer tube is formed from an opaque material.

17. The surgical access device according to claim 14, wherein the light source emits ultraviolet light.

18. The surgical access device according to claim 14, wherein the light source includes a plurality of light emitting elements equidistantly spaced from each other circumferentially about the cavity.

19. The surgical access device according to claim 14, wherein the instrument housing includes a valve assembly disposed within the cavity and the light source is disposed distal to the valve assembly.

20. The surgical access device according to claim 14, wherein the instrument housing includes a power pack coupled thereto, the power pack including a power source and a circuit board, the power pack electrically coupled to the light source to power the light source.
